(19) United States
(12) Patent Application Publication
(10) Pub. No.: US 2004/0034016 A1
(43) Pub. Date: Feb. 19, 2004

(54) Diuretic or sulphonylurea for use in antiviral treatment

(76) Inventors: Ian Stuart Pardoe, Moseley Birmingham (GB); Christopher Edward Hartley, Halesowen (GB)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/380,886

(22) PCT Filed: Sep. 21, 2001
(86) PCT No.: PCT/GB01/04206

(30) Foreign Application Priority Data
Sep. 21, 2000 (GB) 0023199.3
Feb. 19, 2001 (GB) 0104030.2
Feb. 19, 2001 (GB) 0104031.0
Jun. 19, 2001 (GB) 0114947.5

(51) Int. Cl. A61K 31/549; A61K 31/175

(52) U.S. Cl. 514/223.5; 514/592

(57) ABSTRACT

A diuretic, e.g., loop diuretic or thiazide, or a sulphylurea is useful in the treatment of DNA viral infections.

The invention relates to anti-viral treatments and in particular to prophylactic and therapeutic treatments of DNA viral infections such as Herpes virus infections.

Herpes viruses are DNA viruses, having a central core of DNA within a proteinaceous structure. DNA carries the genetic code to reproduce the virus. Viruses must infect a living cell to reproduce. There are numerous viral proteins that are well characterised including important enzymes which act as ideal targets for antiviral chemotherapy. These include DNA polymerase and thymidine kinase which are needed for DNA replication. The replication of viral DNA is essential for virus infectivity. It is known that infecting viruses can alter the natural ionic balances of a living cell in the course of their replication.

We have discovered that certain classes of known drugs can be used for an antiviral effect against DNA viruses.

According to this invention in one aspect there is provided the use of a diuretic or sulphonylurea in the treatment of DNA viral infections acting to alter the natural ionic balance of a living cell to a level less than that which will affect cellular metabolism detrimentally but sufficient to inhibit replication of viral DNA.

The diuretic may be selected from a range of loop diuretics and thiazides.

Recent evidence suggests that the major biotransformation product of frusemide is a glucuronide. Frusemide is extensively bound to plasma proteins, mainly albumin. Plasma concentrations ranging from 1 to 400 mcg/ml are 91-99% bound in healthy individuals. The unbound fraction ranges between 2.3-4.1% at therapeutic concentrations. The terminal half life of frusemide is approximately 2 hours, and it is predominantly excreted in the urine.

Thiazide diuretics include the benzothiadriazines derivatives, also known as thiazides. Typical examples are:

| | |
|---|---|
| althiazide | hydrobenzthiazide |
| bemetizide | hydrochlorothiazide |
| bendroflumethiazide | hydrofluoromethiazide |
| benzthiazide | indapamide |
| benzylhydrochlorothiazide | mebutizide |
| buthiazide | methylcyclothiazide |
| chlorothiazide | meticane |
| chlorothalidone | metalazone |
| cyclopenthiazide | paraflutizide |
| cyclothiazide | polythiazide |
| epithiazide | quinethazone |
| ethiazide | teclothiazide |
| fenquizone | trichlormethiazide |

Preferably the thiazide diuretic is one or more of chlorothiazide, hydrochlorothiazide, hydroflumethiazide, methyclothiazide, trichlormethazide, benzthiazide, bendroflumethazide, bendrofluazide, polythiazide or cyclothiazide.

Sulphonylureas are anti-diabetic drugs which influence ion transport across cell membranes. They are instanced by:

| | |
|---|---|
| acetohexamide | glyburide |
| 1-butyl-3-metanilylurea | glybuthiazole |
| carbutamide | glybuzole |
| chlorpropamide | glycycloamide |
| glibenclamide | glyclopyramide |
| glibornuride | glyhexamide |
| gliclazide | glymidine |
| glimepiride | glypinamide |
| glipizide | phenbutamide |
| gliquidone | tolazamide |
| glisentide | tolbutamide |
| glisolamide | tolcylamide |
| glisoxepid | |

Preferably the sulphonylurea is one or more of tolbutamide, tolazamide, tolcyclamide, glibomuridum, acetohexamide, chlorpropamide, carbutamide, glyburide or glipizide.

By altering the cellular concentrations of ions, cellular ionic balances, cellular ionic milieu and cellular electrical potentials by the application of a diuretic or a sulphonylurea it is possible to change the metabolism of the cell without detriment to the cell but so that virus replication is inhibited. Anti-viral efficacy has been demonstrated against the DNA viruses Herpes simplex virus type 1 and type 2, Feline Herpes virus, Cyclomegalo virus, Varicella zoster virus and Pseudorabies and Adenoviruses. The invention is equally of value in any other intracellular infection such as a bacterial infection as in Chlamydia.

In another aspect the invention provides a composition useful for the treatment of virus infections in subjects, comprising an effective anti-viral amount of a diuretic or sulphonylurea and a suitable carrier.

The compositions of the invention may be adapted for external or internal administration. The formulations may be adapted for slow release. Topical and systemic applications are likely to be the most useful. Other ingredients may be present, provided that they do not compromise the anti-viral activity.

A preferred concentration of loop diuretic is 300 μg in a liquid carrier.

A preferred concentration of thiazide diuretic is from about 0.01 mg/ml to 5.0 mg/ml in a liquid carrier.

A preferred concentration of sulphonylurea is from about 0.5 mg/ml and about 5 mg/ml in a liquid carrier.

A preferred embodiment of this invention is the use of local concentrations of a loop diuretic or sulphonylurea as a highly effective treatment of virus infections of the eye. Recurrent Herpes infections of the cornea in man is the most common viral cause of blindness.

The use of contact lenses carrying e.g. impregnated with a diuretic or sulphonylurea would be a safe and efficient method for creating high intracellular concentrations to prevent or treat the disease. A depot application applied intra-occularly would be a suitable method for the treatment of cytomegalovirus retinitis, a major cause of blindness in patients suffering with AIDS.

It is also within the scope of this invention to provide a combination of one or more of a loop diuretic, a thiazide, a sulphonylurea with or without lithium to produce a synergistic effect.

In order that the invention may be well understood it will now be described by way of illustration only with reference to the following examples:

### EXAMPLE I

In vitro bioassays were undertaken to follow the anti-viral activity of a diuretic compound.

The compositions of frusemide and a carrier were applied to African green monkey kidney and BHK1 veros cells infected with type 2 herpes simplex virus (strains 3345 and 180) at low, intermediate, and high multiplicities of infection (MOI). Inhibition of virus replication was scored on the scale:

| | |
|---|---|
| no inhibition | − |
| 20% inhibition | + |
| 40% inhibition | ++ |
| 60% inhibition | +++ |
| 80% inhibition | ++++ |
| 100% inhibition | +++++ |

T denotes drug toxicity.

The following results were obtained using African green monkey kidney cells and type 2 herpes simplex strain 3345:

Inhibition of hsv2

| Multiplicity of infection (Dose of virus) | Effect of frusemide |
|---|---|
| High | − |
| Medium | ++ |
| Low | ++ |

The experiment was repeated using BHK1 vero cells and type 2 herpes simplex strain 180. Similar results were obtained.

These results show the antiviral effect of frusemide at 1 mg/ml.

### EXAMPLE II

In vitro bioassays were undertaken to determine the anti-viral activity of a thiazide diuretic compound, in the method of Example I.

Inhibition of hsv2

| | |
|---|---|
| | Effect of bendrofluazide |
| | (0.25 mg bendrofluazide/ml |
| Multiplicity of infection (Dose of virus) | liquid medium) |

| | |
|---|---|
| High | ++ |
| Medium | ++++ |
| Low | ++++ |

The experiment was repeated using BHK1 vero cells and type 2 herpes simplex strain 186. Similar results were obtained.

### EXAMPLE III

In vitro bioassays were undertaken to follow the anti-viral activity of a sulphonylurea compound, in the method of Example I.

The following results show the effect of a range of concentrations of tolbutamide over a range of multiplicities of infection using hsv2:

| | 50 mg | 5 mg | 0.5 mg | 0 mg |
|---|---|---|---|---|
| | tolbutamid/ | tolbutamid/ | tolbutamid/ | tolbutamide/ |
| | ml liquid | ml liquid | ml liquid | ml |
| | medium | medium | medium | tolbutamide |
| High | +++ | ++ | + | − |
| multiplicity of | | | | |
| infection | | | | |
| Medium | ++++ | +++ | + | − |
| multiplicity of | | | | |
| infection | | | | |
| Low | +++++ | +++ | ++ | − |
| multiplicity | | | | |
| of infection | | | | |

The experiment was repeated using BHK1 vero cells and type 2 herpes simplex strain 186. Similar results were obtained.

1. Use of a diuretic or sulphonylurea in the treatment of DNA viral infections in living cells to alter the natural balance of the cell to a level less than that which will affect the cellular metabolism detrimentally but sufficient to inhibit replication of viral DNA.

2. Use according to claim 1, wherein the diuretic is a loop diuretic.

3. Use according to claim 2, wherein the loop diuretic is one or more of frusemide, bumetamide, ethacymic acid or torasemide.

4. Use according to claim 3, wherein the loop diuretic is frusemide.

5. Use according to claim 1, wherein the diuretic is a thiazide diuretic.

6. Use according to claim 5, wherein the thiazide diuretic is one or more of chlorothiazide, hydrochlorothiazide, hydroflumethiazide, methyclothiazide, trichlormethazide, benzthiazide, bendroflumethazide, bendrofluazide, polythiazide or cyclothiazide.

7. Use according to claim 1, wherein the sulphonylurea is one or more of tolbutamide, tolazamide, tolcyclamide, glibomuridum, acetohexamide, chlorpropamide, carbutamide, glyburide or glipizide.

8. A composition useful for the treatment of DNA virus infections in subjects, comprising an effective anti-viral amount of a diuretic or sulphonylurea and a suitable carrier.

9. A composition according to claim 8 adapted for topical application.

10. A composition according to claim 8 adapted for systemic application.

11. A composition according to claim 8 adapted for intra-occular depot application.

12. A composition according to any of claims 8 to 11, wherein the diuretic is a loop diuretic.

13. A composition according to claim 12, wherein the loop diuretic is one or more of frusemide, bumetamide, ethacrynic acid or torasemide.

14. A composition according to any of claims 8 to 11, wherein the diuretic is a thiazide diuretic.

15. A composition according to claim 14, wherein the thiazide diuretic is one or more of chlorothiazide, hydrochlorothiazide, hydroflumethiazide, methyclothiazide, trichlormethazide, benzthiazide, bendroflumethazide, bendrofluazide, polythiazide or cyclothiazide.

16. A composition according to any of claim 11, wherein the sulphonylurea is one or more of tolbutamide, tolazamide, tolcyclamide, glibomuridum, acetohexamide, chlorpropamide, carbutamide, glyburide or glipizide.

17. Contact lenses carrying e.g. impregnated, with a diuretic or sulphonylurea.
